(19) United States
(12) Patent Application Publication
(10) Pub. No.: US 2021/0353557 A1
(43) Pub. Date: Nov. 18, 2021

(54) ORAL COMPOSITION CONTAINING REDUCED COENZYME Q10, METHOD FOR PRODUCING SAME, METHOD FOR PREVENTING DISCOLORATION AND DISCOLORATION PREVENTING AGENT

(71) Applicant: SUNTORY HOLDINGS LIMITED, Osaka-shi, Osaka (JP)

(72) Inventors: Yosuke Sato, Soraku-gun, Kyoto (JP); Hikaru Arai, Soraku-gun, Kyoto (JP); Yoshihiro Sakaguchi, Soraku-gun, Kyoto (JP)

(73) Assignee: SUNTORY HOLDINGS LIMITED, Osaka-shi, Osaka (JP)

(21) Appl. No.: 17/287,317

(22) PCT Filed: Oct. 16, 2019
(86) PCT No.: PCT/JP2019/040584

§ 371 Date: Apr. 21, 2021

(30) Foreign Application Priority Data
Oct. 26, 2018 (JP) 2018-202060

(51) Int. Cl. A61K 31/122; A61K 47/22; A61K 47/18; A61K 9/00

(52) U.S. Cl. CPC A61K 31/122; A61K 9/0053; A61K 47/183; A61K 47/22

(57) ABSTRACT

The present invention aims to provide, for example, an oral composition containing reduced CoQ10 that exhibits reduced discoloration caused over time, that has high safety, and that is applicable to foods and drinks, a method for producing the same, and a method for reducing discoloration of a composition containing reduced CoQ10. The present invention relates to, for example, an oral composition containing reduced coenzyme Q10 and at least one sesamin compound, and having a mixing ratio by weight of the sesamin compound to the reduced coenzyme Q10 (sesamin compound/reduced coenzyme Q10) of 0.01 to 10.

### TECHNICAL FIELD

The present invention relates to an oral composition that contains reduced coenzyme Q10 and that exhibits reduced discoloration, and relates to a method for producing the same. The present invention also relates to, for example, a method for reducing discoloration of a composition containing reduced coenzyme Q10 and a discoloration reducing agent.

### BACKGROUND ART

Coenzyme Q10 (hereinafter, also referred to as “CoQ10”) is a benzoquinone derivative widely distributed in nature. CoQ10 is an essential substance as a coenzyme when mitochondria of animal cells produce energy, and is known as a vitamin-like active substance having an effect of improving oxygen use efficiency. Main pharmacological effects of CoQ10 include an antioxidative effect, an energy production promoting effect, an effect of enhancing cardiopulmonary functions, an immunostimulatory effect, and an antiaging effect. CoQ10 is also utilized as a component of health foods, for example.

CoQ10 exists in an oxidized form and a reduced form. Oxidized CoQ10 (molecular formula: C₅₉H₉₀O₄, molecular weight: 863.365) is also referred to as ubiquinone 10. Reduced CoQ10 (molecular formula: C₅₉H₉₂O₄, molecular weight: 865.381) is also referred to as ubiquinol 10, which is a two-electron reduced form of oxidized CoQ10. Oxidized CoQ10 is in the form of yellow to orange crystalline powder, while reduced CoQ10 is in the form of white to pale yellow crystalline powder.

CoQ10 is a nutrient contained in meat and egg at approximately several micrograms per gram and also synthesized in the body, but the amount thereof synthesized in the body decreases with age and the body tends to lack CoQ10. Unfortunately, very few kinds of natural food materials are CoQ10-rich, which makes it difficult to take CoQ10 in an amount that compensates for the deficiency only from everyday meals. To prevent heart disorders common to aged persons, such as ischemic heart disease, or to alleviate the symptoms thereof, supplementing CoQ10 from dietary supplements or fortified foods is desired.

These dietary supplements and fortified foods conventionally contain oxidized CoQ10. Unfortunately, oxidized CoQ10 is less absorptive in oral intake. In contrast, reduced CoQ10 exhibits better oral absorbability than oxidized CoQ10 and is a compound useful for oral compositions such as common foods and drinks, health foods, feed, veterinary drugs, and oral drugs.

Reduced CoQ10 is known to change in tone due to oxygen or light. In particular, mixing reduced CoQ10 into oral compositions such as foods and drinks causes an issue. Patent Literature 1 discloses a solvent selected from hydrocarbons, fatty acid esters, ethers, and nitriles as a compound having an effect of stabilizing reduced CoQ10.

### CITATION LIST

### Patent Literature

### Patent Literature 1: WO 03/006408

### SUMMARY OF INVENTION

### Technical Problem

Discoloration of oral compositions such as foods and drinks, even when having no influence on the efficacy or flavor, may cause worries for the quality. Thus, techniques for reducing discoloration over time due to reduced CoQ10 are important for products containing reduced CoQ10, particularly oral compositions such as foods and drinks. However, the solvent disclosed in Patent Literature 1 itself or a byproduct thereof, when mixed into oral compositions, causes worries for the safety of the bodies. This situation creates a demand for a technique that can more safely reduce discoloration of compositions containing reduced CoQ10 and is applicable to foods and drinks, for example.

The present invention aims to provide an oral composition containing reduced CoQ10 that exhibits reduced discoloration caused over time, that has high safety, and that is applicable to foods and drinks, and to provide a method for producing the same. The present invention also aims to provide, for example, a method for reducing discoloration of a composition containing reduced CoQ10.

### Solution to Problem

The present inventors found that mixing reduced CoQ10 with a sesamin compound can reduce discoloration over time of a composition containing reduced CoQ10. The present inventors also found that theanine is useful for reducing discoloration over time of a composition containing reduced CoQ10.

Specifically, although not limited thereto, the present invention encompasses the following oral composition and the like.

[1] An oral composition containing: reduced coenzyme Q10; and at least one sesamin compound, the oral composition having a mixing ratio by weight of the sesamin compound to the reduced coenzyme Q10 (sesamin compound/reduced coenzyme Q10) of 0.01 to 10.

[2] The oral composition according to the above item [1], further containing theanine.

[3] The oral composition according to the above item [1] or [2], wherein the oral composition has a mixing ratio by weight of the reduced coenzyme Q10 (X), the sesamin compound (Y), and theanine (Z) within a range surrounded by the following points in a three phase diagram:

(X, Y, Z)=(25, 75, 0), (25, 1, 74), (99, 1, 0).

[4] The oral composition according to any one of the above items [1] to [3],

wherein the oral composition has a mixing ratio by weight of the reduced coenzyme Q10 (X), the sesamin compound (Y), and theanine (Z) within a range surrounded by the following points in a three phase diagram:

(X, Y, Z)=(25, 75, 0), (25, 1, 74), (75, 1, 24), (67, 33, 0).

[5] The oral composition according to any one of the above items [1] to [4],

wherein the sesamin compound includes at least one of sesamin and episesamin.

[6] A method for producing an oral composition that contains reduced coenzyme Q10 and that exhibits reduced discoloration, the method including mixing reduced coenzyme Q10 with at least one sesamin compound to give a mixing ratio by weight of the sesamin compound to the reduced coenzyme Q10 (sesamin compound/reduced coenzyme Q10) of 0.01 to 10.

[7] The method for producing an oral composition according to the above item [6], further including mixing the reduced coenzyme Q10 with theanine.

[8] The method for producing an oral composition according to the above item [6] or [7], wherein the reduced coenzyme Q10 is mixed with the at least one sesamin compound or with theanine and the at least one sesamin compound to give a mixing ratio by weight of the reduced coenzyme Q10 (X), the sesamin compound (Y), and the theanine (Z) in the oral composition within a range surrounded by the following points in a three phase diagram:

(X, Y, Z)=(25, 75, 0), (25, 1, 74), (99, 1, 0).

[9] A method for reducing discoloration of a composition containing reduced coenzyme Q10, the method including mixing reduced coenzyme Q10 with at least one sesamin compound.

[10] The method for reducing discoloration of a composition containing reduced coenzyme Q10 according to the above item [9], further including mixing the reduced coenzyme Q10 with theanine.

[11] A discoloration reducing agent for a composition containing reduced coenzyme Q10, containing at least one sesamin compound as an active ingredient.

[12] The discoloration reducing agent according to the above item [11], further containing theanine as an active ingredient.

[13] Use of at least one sesamin compound for reducing discoloration of a composition containing reduced coenzyme Q10.

[14] The use according to the above item [13], further including use of theanine.

### Advantageous Effects of Invention

The present invention provides, for example, an oral composition containing reduced CoQ10 that exhibits reduced discoloration over time, that has high safety, and that is applicable to foods and drinks, and a method for producing the same. The present invention can reduce discoloration of a composition containing reduced CoQ10.

### BRIEF DESCRIPTION OF THE DRAWINGS

FIG. 1 is a three phase diagram of the proportions (% by weight) of reduced coenzyme Q10 (X), a sesamin compound (Y), and theanine (Z) with the sum of the amounts taken as 100% by weight.

### DESCRIPTION OF EMBODIMENTS

The oral composition of the present invention contains reduced coenzyme Q10 (reduced CoQ10) and at least one sesamin compound and has a mixing ratio by weight of the sesamin compound to the reduced coenzyme Q10 (sesamin compound/reduced coenzyme Q10) of 0.01 to 10.

Reduced CoQ10 is white to pale yellow, and discolors into yellow to orange due to oxygen or light. The present invention is such that reduced CoQ10 is mixed with at least one sesamin compound to reduce discoloration (change in tone) over time of a composition containing reduced CoQ10.

Discoloration of a composition containing reduced coenzyme Q10 may be examined by determining the change in tone over time of the composition based on the ΔE value (color difference) calculated using a color difference meter. For example, discoloration may be examined by storing a composition for a certain period of time and then calculating the ΔE value (color difference) between the composition before storage and the composition after storage with a color difference meter. The smaller the ΔE value is, the lower the degree of discoloration is. It is considered as a reduce of discoloration. The ΔE value may be calculated by the following formula with the L values, a values, and b values of the composition before storage and the composition after storage being measured using a color difference meter.

ΔE={(ΔL)²+(Δa)²+(Δb)²}^{1/2}

ΔL=(L value before storage)−(L value after storage), Δa=(a value before storage)−(a value after storage), Δb=(b value before storage)−(b value after storage)

As will be described in the EXAMPLES below, a sample containing reduced CoQ10 showed an increase in the ΔE value (color difference) over time. Mixing reduced CoQ10 with a sesamin compound reduced an increase in the ΔE value over time, reducing discoloration. Sesamin compounds are highly safe substances that are taken as foods and drinks from old.

“Sesamin compound” is a general term for compounds including sesamin and analogs thereof. Sesamin is one of main lignan compounds contained in sesame. Examples of the sesamin compound to be used in the present invention include sesamin, episesamin, sesaminol, episesamin, episesaminol, sesamolin, 2-(3,4-methylenedioxyphenyl)-6-(3-methoxy-4-hydroxyphenyl)-3,7-dioxabicyclo[3,3,0]octane, 2,6-bis-(3-methoxy-4-hydroxyphenyl)-3,7-dioxabicyclo[3,3,0]octane, and 2-(3,4-methylenedioxyphenyl)-6-(3-methoxy-4-hydroxyphenoxy)-3,7-dioxabicyclo[3,3,0]octane. Each of these may be used alone or two or more of these may be used in combination. To sufficiently achieve the effects of the present invention, the sesamin compound used preferably contains sesamin and/or episesamin, more preferably contains sesamin and episesamin. In the case of using sesamin and episesamin, the sesamin:episesamin ratio (by weight) is preferably, but not limited to, 1:0.5 to 1:1.5, more preferably 1:0.7 to 1:1.3.

The sesamin compound to be used in the present invention is not limited, for example, by its form or production method. For example, the sesamin compound used may also be an extract (referred to as an extract or purified product rich in sesamin compound) obtainable from sesame oil by a known method, such as a method including adding hot methanol to sesame oil to provide an extract, removing the methanol, and adding acetone to the residue to further provide an extract (disclosed in JP H04-009331 A). The sesamin compound is also obtainable by synthesis. A method for synthesis, in the case of sesamin and episesamin, may be the method by Beroza et al. (J. Am. Chem. Soc., 78, 1242 (1956)).

The oral composition of the present invention has a mixing ratio by weight of the sesamin compound to the reduced CoQ10 (sesamin compound/reduced CoQ10) of 0.01 to 10. A mixing ratio by weight of the sesamin compound to the reduced CoQ10 within the above range can reduce discoloration of the composition containing reduced CoQ10. To reduce discoloration of the composition, the mixing ratio by weight of the sesamin compound to the reduced CoQ10, sesamin compound/reduced CoQ10, is preferably 0.05 or higher, more preferably 0.1 or higher, still more preferably 0.3 or higher. From the viewpoint of economic efficiency, for example, the upper limit of the mixing ratio by weight of the sesamin compound to the reduced CoQ10 (sesamin compound/reduced CoQ10) is preferably 5 or lower, more preferably 3 or lower. In an embodiment, to reduce discoloration of the composition, for example, the mixing ratio by weight of the sesamin compound to the reduced CoQ10 (sesamin compound/reduced CoQ10) is preferably 0.05 to 5, more preferably 0.1 to 3, still more preferably 0.3 to 3. In another embodiment, the mixing ratio by weight of the sesamin compound to the reduced CoQ10 (sesamin compound/reduced CoQ10) may be 0.01 to 0.1, or may be 0.01 or higher and lower than 0.1. The mixing ratio by weight of the sesamin compound to the reduced CoQ10, sesamin compound/reduced CoQ10, is the ratio of the amount (weight) of the sesamin compound mixed to the amount (weight) of the reduced CoQ10 mixed. The amount of the sesamin compound mixed means the sum of the amounts of the sesamin compounds mixed. In other words, in the case of using two or more compounds as sesamin compounds, the amount of the sesamin compound mixed is the sum of the amounts thereof.

To reduce discoloration of the composition, the oral composition of the present invention may contain theanine. Theanine is one of amino acids and is known to be contained in green tea, for example. The theanine to be used in the present invention is not limited, for example, by its origin or production method. In an embodiment of the present invention, the presence of theanine and at least one sesamin compound can more reduce discoloration of an oral composition containing reduced CoQ10. To reduce discoloration of the composition, the oral composition, when containing theanine, preferably has a mixing ratio by weight of the theanine to the reduced CoQ10 (theanine/reduced CoQ10) of 0.2 or higher, more preferably 0.3 or higher, still more preferably 0.5 or higher, while preferably 3.5 or lower, more preferably 3 or lower. To reduce discoloration of the composition, the mixing ratio by weight of the theanine to the reduced CoQ10 (theanine/reduced CoQ10) is preferably 0.2 to 3.5, more preferably 0.3 to 3, still more preferably 0.5 to 3, for example. The mixing ratio by weight of the theanine to the reduced CoQ10 (theanine/reduced CoQ10) is the ratio of the amount (weight) of the theanine mixed to the amount (weight) of the reduced CoQ10 mixed.

Theanine is also known to have an effect of controlling autonomic nerves, and is expected to further add beneficial functions to an oral composition containing reduced CoQ10 and a sesamin compound.

When the oral composition contains reduced CoQ10, a sesamin compound and theanine, it preferably has a mixing ratio by weight of the sum of the sesamin compound and the theanine to the reduced CoQ10 (sum of sesamin compound and theanine/reduced CoQ10) of 0.3 to 10, more preferably 0.4 to 5, still more preferably 0.5 to 5, for example, to reduce discoloration of the composition, for example. In an embodiment, the mixing ratio by weight of the theanine to the sesamin compound (theanine/sesamin compound) may be 0.5 to 80 or 0.5 to 75.

In an embodiment, in order to reduce discoloration of the composition, the mixing ratio by weight of the reduced CoQ10 (X), the sesamin compound (Y), and the theanine (Z) in the oral composition preferably falls within a range surrounded by the following points in a three phase diagram. The range surrounded by the following points includes the following points and the points on the line segments connecting the following points. The same applies to the ranges surrounded by other points.

### (X, Y, Z)=(25, 75, 0), (25, 1, 74), (99, 1, 0)

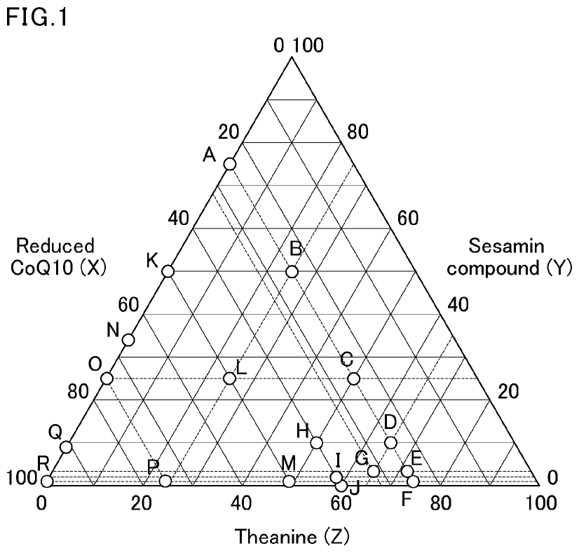

In the present invention, the three phase diagram is a three phase diagram that indicates the mixing ratio by weight (proportions (% by weight)) of the reduced CoQ10 (X), the sesamin compound (Y), and the theanine (Z) with the sum of the amounts (weight) of the respective components being taken as 100 (100% by weight). As described above, the amount of the sesamin compound (Y) mixed means the sum of the amounts of the sesamin compounds mixed. The mixing ratio by weight of the sesamin compound (Y) can be determined from the sum of the amounts of the sesamin compounds mixed. FIG. 1 shows a three phase diagram of the mixing ratio by weight (proportions (% by weight)) of the reduced CoQ10 (X), the sesamin compound (Y), and the theanine (Z) with the sum of the amounts of the respective components being taken as 100 (% by weight). The ratios A to R in FIG. 1 are examples of the ratio of the components in the oral composition of the present invention. The coordinates (X, Y, Z) of each of the ratios A to R in FIG. 1 are shown below.

A(25, 75, 0), B(25, 50, 25), C(25, 25, 50), D(25, 10, 65), E(25, 2, 73), F(25, 1, 74), G(32, 3, 65), H(40, 10, 50), I(40, 2, 58), J(40, 1, 59), K(50, 50, 0), L(50, 25, 25), M(50, 1, 49), N(67, 33, 0), O(75, 25, 0), P(75, 1, 24), Q(91, 9, 0), R(99, 1, 0)

In the case where the mixing ratio by weight of the reduced CoQ10 and the sesamin compound, as well as theanine optionally mixed, falls within the range surrounded by A, F, and Q in the three phase diagram, an effect of reducing discoloration of the composition containing reduced CoQ10 can be achieved in a range surrounded by any of the points A to Q. In a preferred embodiment of the present invention, the oral composition contains reduced CoQ10 and a sesamin compound or contains reduced CoQ10, a sesamin compound, and theanine in a ratio within the above range. To reduce discoloration of the composition, the oral composition preferably contains theanine as described above. For the composition containing theanine, the mixing ratio by weight of the theanine (Z) is higher than 0 (% by weight) in the three phase diagram. In an embodiment, preferably, the mixing ratio by weight of the reduced CoQ10 (x), the sesamin compound (Y), and the theanine (Z) in the oral composition falls within the range surrounded by A, F, and Q in the three phase diagram and falls within the range where the mixing ratio by weight of the theanine (Z) is higher than 0 (% by weight). The amounts of the reduced CoQ10, the sesamin compound, and the theanine can be quantified by a known method with high performance liquid chromatography (HPLC).

To reduce discoloration of the composition, the mixing ratio by weight of the reduced CoQ10 (X), the sesamin compound (Y), and the theanine (Z) more preferably falls within the range surrounded by the following points in the three phase diagram.

(X, Y, Z)=(25, 75, 0), (25, 1, 74), (75, 1, 24), (67, 33, 0)

The range surrounded by the above points corresponds to the range surrounded by A, F, P, and N in the three phase diagram of FIG. 1. The mixing ratio by weight of the reduced CoQ10 (X), the sesamin compound (Y), and the theanine (Z) still more preferably falls within the range surrounded by the following points in the three phase diagram.

(X, Y, Z)=(25, 75, 0), (25, 1, 74), (75, 1, 24), (50, 50, 0)

The range surrounded by the above points corresponds to the range surrounded by A, F, P, and K in the three phase diagram. In a preferred embodiment, the oral composition contains theanine. In an embodiment, preferably, the mixing ratio by weight of the reduced CoQ10 (X), the sesamin compound (Y), and the theanine (Z) falls within the range surrounded by A, F, P, and K in the three phase diagram and falls within the range where the mixing ratio by weight of the theanine (Z) is higher than 0 (% by weight).

In an embodiment, the mixing ratio by weight of the reduced CoQ10 (X), the sesamin compound (Y), and the theanine (Z) particularly preferably falls within any of the ranges surrounded by the following points in the three phase diagram:

(X, Y, Z)=(25, 50, 25), (25, 1, 74), (50, 1, 49), (50, 25, 25);

(X, Y, Z)=(25, 50, 25), (25, 1, 74), (40, 1, 59), (50, 25, 25);

(X, Y, Z)=(25, 50, 25), (25, 1, 74), (40, 1, 59), (40, 10, 50);

(X, Y, Z)=(25, 50, 25), (25, 1, 74), (40, 1, 59);

(X, Y, Z)=(25, 50, 25), (25, 1, 74), (32, 3, 65); or

(X, Y, Z)=(25, 25, 50), (25, 1, 74), (32, 3, 65).

In an embodiment, the mixing ratio by weight of the reduced CoQ10 (X), the sesamin compound (Y), and the theanine (Z) may fall within the range surrounded by the following points in the three phase diagram.

### (X, Y, Z)=(25, 1, 74), (99, 1, 0), (91, 9, 0)

The range surrounded by the above points corresponds to the range surrounded by F, R, and Q in the three phase diagram.

The mixing ratio by weight of the reduced CoQ10 (X), the sesamin compound (Y), and the theanine (Z) may fall within the range surrounded by F, R, and Q in the three phase diagram, except for (X, Y, Z)=(91, 9, 0) (Q).

In the present invention, any of optional components and additives may be mixed in addition to the reduced CoQ10 and the sesamin compound as well as theanine that is optionally mixed if desired, unless the effects thereof are impaired. Examples of optional components and additives include those commonly used for oral compositions such as foods and drinks, oral drugs, and feed. Examples thereof include physiologically active substances such as vitamins, e.g., vitamin E and vitamin C, minerals, and nutrient components, and those mixed in drug formulation such as vehicles, binders, emulsifiers, tonicity agents (isotonizing agents), buffers, dissolution aids, antiseptics, stabilizing agents, antioxidants, colorants, coagulating agents, coating agents, and flavoring agents. One of these may be used or two or more of these may be used in combination. In addition to the above, other components may be mixed as appropriate such as materials used for oral compositions, e.g., foods and drinks, oral drugs, and feed in accordance with the use thereof.

The oral composition of the present invention may contain reduced CoQ10 in any proportion. For example, reduced CoQ10 may be mixed such that the sum of reduced CoQ10 and a sesamin compound represents 10 to 100% by weight in the oral composition. In an embodiment, reduced CoQ10 may be mixed such that the sum of reduced CoQ10, a sesamin compound, and theanine represents 10 to 100% by weight in the oral composition, for example.

The oral composition may be any of foods and drinks, oral drugs, and feed. Drugs may be either human drugs or nonhuman, veterinary drugs. Foods and drinks include common foods and drinks, health foods, foods with functional claims, foods for specified health uses, foods for the sick, food additives, and materials thereof. The oral composition may be in any dosage form or shape, such as a solid form, e.g., powder, a granule, a tablet, a capsule (including a soft capsule and a hard capsule), or a chewable agent; a liquid form, e.g., liquid, suspension, or emulsion; or a semisolid form such as paste. The oral composition of the present invention may be used as an inner composition of a capsule.

Reduced CoQ10 and sesamin compounds are soluble in fats. In an embodiment, the oral composition of the present invention containing reduced CoQ10 and at least one sesamin compound is preferably taken in the form of solution, emulsion, or dispersion in oil or fat in order to efficiently take the reduced CoQ10 and the at least one sesamin compound. From this viewpoint, the oral composition of the present invention also preferably contains oil or fat. The oil or fat to be used may be any one that can dissolve the sesamin compound and reduced CoQ10. Examples thereof include oils which are liquid at room temperature such as natural animal or vegetable oils, e.g., soybean oil, rapeseed oil, safflower oil, olive oil, cottonseed oil, corn oil, palm oil, rice bran oil, wheat germ oil, sunflower oil, safflower oil, peanut oil, whale oil, sardine oil, and cuttlefish oil; and oils such as medium-chain fatty acid triglycerides, mono-, di-, or tri-glycerides of C4-C22 fatty acids, and mixtures of any of these. Examples also include mixture of any of the above oils which are liquid at room temperature with any of fats which are semisolid or solid at room temperature such as lard, beef tallow, hydrogenated fish oil, margarine, and shortening.

In an embodiment where the oral composition is for example a health food containing reduced CoQ10 and at least one sesamin compound as active ingredients, the reduced CoQ10 and the sesamin compound may be dissolved, emulsified, or dispersed in the fat or oil so that the composition may be encapsulated into a microcapsule, a soft capsule, or a hard capsule. Such encapsulation enables drug formulation for the purpose of nutrition supply or auxiliary supply of nutrient components, resulting in efficient intake of reduced CoQ10. Further, since reduced CoQ10 is photodegradable, it is preferable to protect the composition containing reduced CoQ10 from light and reduce discoloration of the composition by, for example, encapsulating the composition containing reduced CoQ10 in a capsule formed from a capsule material mixed with coloring matter such as caramel coloring or titanium dioxide.

The oral composition of the present invention may be produced by any method, for example by mixing reduced CoQ10 with a sesamin compound to give a mixing ratio by weight of the sesamin compound to the reduced coenzyme Q10 (sesamin compound/reduced coenzyme Q10) of 0.01 to 10.

The present invention encompasses a method for producing an oral composition including mixing reduced CoQ10 with at least one sesamin compound to give a mixing ratio by weight of the sesamin compound to the reduced coenzyme Q10 (sesamin compound/reduced coenzyme Q10) of 0.01 to 10. The production method of the present invention can provide an oral composition that contains reduced CoQ10 and exhibits reduced discoloration. The sesamin compound and preferred embodiments thereof are the same as those described above. In the present invention, theanine is preferably further mixed. Mixing reduced coenzyme Q10 with theanine and at least one sesamin compound can further reduce discoloration of the resulting composition.

A preferred range of the mixing ratio by weight of the reduced CoQ10, the sesamin compound, and the theanine optionally mixed as desired in the oral composition is the same as that for the aforementioned oral composition.

In the production method of the present invention, the reduced CoQ10 and the sesamin compound are preferably mixed to give a mixing ratio by weight of the sesamin compound to the reduced CoQ10 (sesamin compound/reduced CoQ10) within the aforementioned range. Specifically, in an embodiment, for example, these components are preferably mixed to give a mixing ratio by weight of the sesamin compound to the reduced CoQ10 (sesamin compound/reduced CoQ10) of 0.05 to 5, more preferably 0.1 to 3, still more preferably 0.3 to 3. In another embodiment, these components may be mixed to give a mixing ratio by weight of the sesamin compound to the reduced CoQ10 (sesamin compound/reduced CoQ10) of 0.01 to 0.1 or 0.01 or higher and lower than 0.1. In an embodiment where theanine is mixed, the theanine is preferably mixed to give a mixing ratio by weight of the theanine to the reduced CoQ10 (theanine/reduced CoQ10) of 0.2 to 3.5, more preferably 0.3 to 3, still more preferably 0.5 to 3.

In addition, in order to for example reduce discoloration of the resulting composition, the reduced CoQ10 is preferably mixed with at least one sesamin compounds or with theanine and at least one sesamin compound to give a mixing ratio by weight of the reduced CoQ10 (x), the sesamin compound (Y), and the theanine (Z) in the oral composition within the range surrounded by the following points in the three phase diagram. More preferred is mixing with theanine and at least one sesamin compound.

### (X, Y, Z)=(25, 75, 0), (25, 1, 74), (99, 1, 0)

To reduce discoloration of the resulting composition, a preferred mixing ratio by weight of the reduced CoQ10, the sesamin compound, and the theanine to be optionally mixed is the same as that for the oral composition. A preferred mixing ratio by weight of the reduced CoQ10 (X), the sesamin compound (Y), and the theanine (Z) also preferably falls within the range surrounded by the following points in the three phase diagram. More preferably, the reduced CoQ10 is mixed with theanine and at least one sesamin compound such that the mixing ratio falls within the range surrounded by the following points and the mixing ratio by weight of the theanine is higher than 0 (% by weight).

### (X, Y, Z)=(25, 75, 0), (25, 1, 74), (75, 1, 24), (67, 33, 0)

In an embodiment, the mixing ratio by weight of the reduced CoQ10 (X), the sesamin compound (Y), and the theanine (Z) may fall within the range surrounded by the points (X, Y, Z)=(25, 1, 74), (99, 1, 0), (91, 9, 0) in the three phase diagram, and may fall within this range except for the point (99, 1, 0).

In the case of mixing any of the aforementioned optional components and additives into the oral composition, the components may be mixed in any manner, such as in any order.

The present invention also encompasses a method for reducing discoloration of a composition containing reduced CoQ10, including mixing reduced CoQ10 with at least one sesamin compound. Mixing a composition containing reduced coenzyme Q10 with at least one sesamin compound can reduce discoloration of the composition.

The sesamin compounds and preferred embodiments thereof are the same as those described above. The method for reducing discoloration of the present invention preferably includes mixing theanine in addition to the at least one sesamin compound. Mixing theanine and at least one sesamin compound into a composition containing reduced coenzyme Q10 can more reduce discoloration of the composition. The mixing ratio by weight of the sesamin compound and the theanine to the reduced CoQ10, a preferred range thereof and the like are the same as those for the aforementioned oral composition. In a preferred embodiment of the composition containing reduced CoQ10, the reduced CoQ10 is mixed with at least one sesamin compound to give a mixing ratio by weight of the reduced CoQ10 and the sesamin compound within the aforementioned range. In a more preferred embodiment of the composition containing reduced CoQ10, the reduced CoQ10 is mixed with at least one sesamin compound or with theanine and at least one sesamin compound, preferably with theanine and at least one sesamin compound, to give a mixing ratio by weight of the reduced CoQ10, the sesamin compound, and the theanine within the aforementioned range.

The present invention can reduce discoloration caused over time of a composition containing reduced CoQ10. The composition containing reduced CoQ10 is preferably an oral composition. The method for reducing discoloration of the present invention is suitably used for reducing discoloration of an oral composition containing reduced CoQ10.

The composition containing reduced CoQ10 may contain any of optional components, additives, and other substances that may be mixed into the aforementioned oral composition.

The present invention also encompasses a discoloration reducing agent for a composition containing reduced coenzyme Q10, the agent containing at least one sesamin compound as active ingredients. The present invention also encompasses use of at least one sesamin compound for reducing discoloration of a composition containing reduced coenzyme Q10. The discoloration reducing agent preferably further contains theanine as an active ingredient. The use preferably further includes use of theanine.

The composition containing reduced CoQ10 is preferably an oral composition. The sesamin compounds and preferred embodiments thereof are the same as those described above. In the discoloration reducing agent and use of the present invention, the mixing ratio by weight of the sesamin compound to the reduced CoQ10 (sesamin compound/reduced CoQ10), the mixing ratio by weight of the reduced CoQ10 (X), the sesamin compound (Y), and the theanine (Z), and preferred ranges thereof, for example, are the same as those for the aforementioned oral composition. In a preferred embodiment of the composition containing reduced CoQ10, the reduced CoQ10 is mixed with at least one sesamin compound to give a mixing ratio by weight of the reduced CoQ10 and the sesamin compound within the aforementioned range. In a more preferred embodiment of the composition containing reduced CoQ10, the reduced CoQ10 is mixed with at least one sesamin compound or with theanine and at least one sesamin compound, preferably with theanine and at least one sesamin compound, to give a mixing ratio by weight of the reduced CoQ10, the sesamin compound, and the theanine within the aforementioned range. The discoloration reducing agent may be mixed with reduced coenzyme Q10 for use, and is preferably used to give a mixing ratio by weight of the reduced coenzyme Q10, the sesamin compound, and the theanine within the above range.

### EXAMPLES

Hereinafter, the present invention is described in more detail with reference to examples. Still, these examples are not intended to limit the scope of the present invention.

In the examples, the following reduced CoQ10, sesamin compound, theanine, and dextrin were used.

Reduced CoQ10: trade name Kaneka QH, available from Kaneka Corp.

Sesamin compounds: mixture of sesamin:episesamin=47:53 (ratio by weight), available from Takemoto Oil & Fat Co., Ltd.

Theanine: trade name Suntheanine, available from Taiyo Kagaku Co., Ltd.

Dextrin: trade name Pinedex #100, available from Matsutani Chemical Industry Co., Ltd.

In the examples, the color difference was measured by the following method.

### <Method for Measuring Color Difference>

The L value, a value, and b value of a sample were measured using a color difference meter (trade name: Spectrophotometer SE7700, available from Nippon Denshoku Industries Co., Ltd.).
The number n of samples was 3 for each standard unless otherwise mentioned. The average values of the three samples were defined as the L value, a value, and b value of the standard.
Measurement was performed five times for each sample, and the average value was output as the result. The sample was stirred for each measurement operation so that the whole sample was subjected to the measurement.
In the measurement, about 1 g of a sample was weighed out from a Petri dish for use. The sample in the Petri dish was mixed and homogenized before weighing out.

### <ΔE Value (Color Difference)>

The ΔE value was calculated by the following formula.

ΔE={(ΔL)²+(Δa)²+(Δb)²}^{1/2}

For each standard after storage for a predetermined period of time, the ΔE value was calculated based on the 0-time value (value after 0-hour storage) of the standard. For a control after storage for a predetermined period of time, the ΔE value was also calculated based on the 0-time value (value after 0-hour storage) of the control.

For example, for a sample of a standard A after storage for one week (1-week sample of standard A), the ΔE value was calculated as follows.

ΔE=[{(0-time L value of standard A)−(1-week L value of standard A)}²+{(0-time a value of standard A)−(1-week a value of standard A)}²+{(0-time b value of standard A)−(1-week b value of standard A)}²]^{1/2}

Lower ΔE value indicates more reduction of discoloration (change in tone).

### <Rate of Change Relative to Control>

For the control and each sample after storage for a predetermined period of time, the percentage of the ΔE value of the sample with the ΔE value of the control taken as 100% is defined as the rate of change (%) relative to control.

For example, in the case of a sample of a standard A after storage for one week (1-week sample of standard A), the rate of change relative to control is calculated as follows.

Rate of change (%) relative to control={(ΔE value of 1-week sample of standard A)/(ΔE value of 1-week sample of control)}×100

Lower rate of change relative to control indicates more reduction of discoloration.

### Reference Example 1

About 2 g each of the reduced CoQ10, sesamin compound, theanine, and dextrin were put into separate Petri dishes (n=1) and left to stand at a temperature of 40° C. and a humidity of 75% RH (accelerated condition). The L value, a value, and b value were measured at the start of storage (0-hour storage) and after 7-day (168-hour) storage, and then the ΔE value was calculated. For each sample, the change in appearance after seven days from the start of storage was visually observed.

The results are shown in Table 1. The sesamin compound, the theanine, and the dextrin (standards S-2 to S-4) showed little change in ΔE value after storage. The reduced CoQ10 (standard S-1) showed an increase in ΔE value over time. Visual observation of the appearance showed that all standards were white at the start of storage. The standard S-1 turned yellow and darkened over time, and finally became orange after seven days. The theanine, the sesamin compound, and the dextrin looked white to pale yellow by visual observation even after seven days. This confirmed that the theanine, the sesamin compound, and the dextrin showed little change in tone after storage.

TABLE 1

| | | Standard | | | |
|---|---|---|---|---|---|
| | | S-1 | S-2 | S-3 | S-4 |
| | | Reduced | Sesamin | | |
| | Compound | CoQ10 | compound | Theanine | Dextrin |
| ΔE | 0-Hour storage | 0.0 | 0.0 | 0.0 | 0.0 |
| | After 7 days | 66.4 | 0.9 | 0.1 | 0.6 |

### Example 1

The materials were weighed, put into a bag, and mixed therein, whereby compositions of three standards shown in Table 2 were prepared. About 2 g each of the compositions were put into separate Petri dishes and left to stand at a temperature of about 20° C. and a humidity of about 50% RH for storage. The L value, a value, and b value of each sample were measured immediately after preparation (at the start of storage: 0-hour storage), after 8-hour storage, after 1-day (24-hour) storage, after 4-day (96-hour) storage, and after 7-day (168-hour) storage, and then the ΔE value was calculated.

Table 3 shows the change in ΔE value over time of each standard. The rate (%) of the ΔE value of the standards 1-2 and 1-3 after 7-day storage was calculated as the rate of change relative to control with the ΔE value of the standard 1-1 (control) after 7-day storage taken as 100%. This rate of change is shown in Table 3.

TABLE 2

| | | Standard 1-1 | Standard 1-2 | Standard 1-3 |
|---|---|---|---|---|
| Formulation | Reduced CoQ10 | 10 | 10 | 10 |
| (g) | Sesamin | 0 | 10 | 10 |
| | compound | | | |
| | Theanine | 0 | 0 | 10 |
| | Dextrin | 20 | 10 | 0 |
| | Sum | 30 | 30 | 30 |

TABLE 3

| | | Standard 1-1 | Standard 1-2 | Standard 1-3 |
|---|---|---|---|---|
| ΔE | Immediately after | 0.0 | 0.0 | 0.0 |
| | preparation | | | |
| | After 8 hours | 1.6 | 1.1 | 1.1 |
| | After 1 day | 4.4 | 3.5 | 2.5 |
| | After 4 days | 21.9 | 18.1 | 11.3 |
| | After 7 days | 37.3 | 31.1 | 23.2 |

| | | | |
|---|---|---|---|
| Rate of change (%) | 100.0 | 83.4 | 62.2 |

The compositions of the standards 1-1 to 1-3 containing the reduced CoQ10 showed an increase in ΔE value over time. Mixing of the sesamin compound or of the sesamin compound and theanine reduced the increase in ΔE value. In other words, mixing of the reduced CoQ10 with the sesamin compound or with the sesamin compound and theanine successfully reduced discoloration (change in tone). In the case of 7-day storage in a Petri dish, the ΔE value of the standard 1-2 in which the reduced CoQ10 was mixed with the sesamin compound was about 6 point lower, and the ΔE value of the standard 1-3 in which the reduced CoQ10 was mixed with the sesamin compound and theanine was about 14 point lower, respectively, than the ΔE value of the standard 1-1 in which the reduced CoQ10 was mixed only with the dextrin.

### Example 2

The experiment was performed under the same conditions as in Example 1, except that the ratio of the reduced CoQ10 and the sesamin put into a Petri dish was changed as shown in Table 4. The L value, a value, and b value of each sample were measured immediately after preparation (0-hour storage) and after 7-day (168-hour) storage, and then the ΔE value was calculated. The ΔE value after 7-day storage is shown in Table 4. For the ΔE value after 7-day storage, the rate (%) of the ΔE value of each of the standards 2-2 to 2-4 relative to the ΔE value of the standard 2-1 (control) was calculated as the rate of change relative to control. This rate of change (%) is also shown in Table 4. The compositions of the standards 2-2 to 2-4 exhibited a reduced change in tone. The mixing ratio by weight of the sesamin compound to the reduced CoQ10 (sesamin compound/reduced CoQ10) was 0.01 in the standard 2-2, 0.1 in the standard 2-3, and 0.5 in the standard 2-4. For example, the standard 2-4 corresponds to the composition indicated by N(67, 33, 0) in the three phase diagram of FIG. 1.

TABLE 4

| | | Standard 2-1 | Standard 2-2 | Standard 2-3 | Standard 2-4 |
|---|---|---|---|---|---|
| Formulation | Reduced CoQ10 | 10.0 | 10.0 | 10.0 | 10.0 |
| (g) | Sesamin compound | 0.0 | 0.1 | 1.0 | 5.0 |
| | Dextrin | 10.0 | 9.9 | 9.0 | 5.0 |
| | Sum | 20.0 | 20.0 | 20.0 | 20.0 |
| Evaluation | ΔE (after 7 days) | 39.1 | 38.0 | 37.3 | 34.6 |
| results | Rate of change (%) | 100.0 | 97.2 | 95.4 | 88.5 |

### Example 3

The materials were weighed, put into a bag, and mixed therein, whereby compositions of respective standards were prepared. The formulations (% by weight) of the compositions of the respective standards are shown in Table 5. About 2 g each of the compositions were put into separate Petri dishes and left to stand under the same conditions as in Example 1 for storage. The L value, a value, and b value of each sample were measured immediately after preparation (0-hour storage), after 1-week storage (7-day storage), after 2-week storage (14-day storage), and after 4-week storage (28-day storage), and then the ΔE value was calculated. In the Evaluation Nos. 1 to 5 shown in Table 5, the ΔE value was calculated based on the same control within the same evaluation group. The standards A to M, O, and P shown in Table 5 are indicated in the three phase diagram of FIG. 1.

TABLE 5

| Eval- | | Formulation (wt %) | | | | |
|---|---|---|---|---|---|---|
| uation | | Reduced | Sesannin | Theanine | | |
| No. | Standard | CoQ10 (X) | compound (Y) | (Z) | Dextrin | Sum |
| 1 | Control 1 | 25 | 0 | 0 | 75 | 100 |
| | A | 25 | 75 | 0 | 0 | 100 |
| | B | 25 | 50 | 25 | 0 | 100 |
| | C | 25 | 25 | 50 | 0 | 100 |
| | D | 25 | 10 | 65 | 0 | 100 |
| | E | 25 | 2 | 73 | 0 | 100 |
| | F | 25 | 1 | 74 | 0 | 100 |
| 2 | Control 2 | 32 | 0 | 0 | 68 | 100 |
| | G | 32 | 3 | 65 | 0 | 100 |
| 3 | Control 3 | 40 | 0 | 0 | 60 | 100 |
| | H | 40 | 10 | 50 | 0 | 100 |
| | I | 40 | 2 | 58 | 0 | 100 |
| | J | 40 | 1 | 59 | 0 | 100 |
| 4 | Control 4 | 50 | 0 | 0 | 50 | 100 |
| | K | 50 | 50 | 0 | 0 | 100 |
| | L | 50 | 25 | 25 | 0 | 100 |
| | M | 50 | 1 | 49 | 0 | 100 |
| 5 | Control 5 | 75 | 0 | 0 | 25 | 100 |
| | O | 75 | 25 | 0 | 0 | 100 |
| | P | 75 | 1 | 24 | 0 | 100 |

A change in ΔE value over time of each sample is shown in Table 6. For the ΔE value of each standard after 2-week storage, the rate of the ΔE value of each standard with the ΔE value of the control taken as 100% was calculated as the rate of change relative to control. This rate of change is also shown in Table 6. The standards A to M, O, and P exhibited a reduced change in tone compared to the control.

TABLE 6

| | | ΔE | | | | Rate of |
|---|---|---|---|---|---|---|
| | | Immediately | | | | change after |
| Evaluation | | after | After | After | After | 2 weeks |
| No. | Standard | preparation | 1 week | 2 weeks | 4 weeks | (%) |
| 1 | Control 1 | 0.0 | 31.4 | 47.1 | 50.9 | 100.0 |
| | A | 0.0 | 27.7 | 37.0 | 37.7 | 78.6 |
| | B | 0.0 | 22.0 | 31.4 | 34.0 | 66.7 |
| | C | 0.0 | 17.5 | 27.7 | 33.6 | 58.8 |
| | D | 0.0 | 21.2 | 29.0 | 39.9 | 61.6 |
| | E | 0.0 | 17.0 | 28.4 | 37.1 | 60.3 |
| | F | 0.0 | 16.7 | 26.6 | 29.0 | 56.5 |
| 2 | Control 2 | 0.0 | 37.1 | 53.2 | 55.5 | 100.0 |
| | G | 0.0 | 18.6 | 31.6 | 40.5 | 59.4 |
| 3 | Control 3 | 0.0 | 35.4 | 53.6 | 58.5 | 100.0 |
| | H | 0.0 | 25.2 | 38.5 | 48.9 | 71.8 |
| | I | 0.0 | 25.6 | 42.2 | 46.2 | 78.7 |
| | J | 0.0 | 24.6 | 39.2 | 48.6 | 73.1 |
| 4 | Control 4 | 0.0 | 37.6 | 60.0 | 65.2 | 100.0 |
| | K | 0.0 | 36.0 | 50.9 | 53.4 | 84.8 |
| | L | 0.0 | 28.8 | 44.7 | 53.5 | 74.5 |
| | M | 0.0 | 27.3 | 46.0 | 51.1 | 76.7 |
| 5 | Control 5 | 0.0 | 39.8 | 63.1 | 69.9 | 100.0 |
| | O | 0.0 | 39.0 | 58.0 | 64.2 | 91.9 |
| | P | 0.0 | 35.9 | 56.6 | 65.5 | 89.7 |

### INDUSTRIAL APPLICABILITY

The present invention is useful in the fields of foods and drinks, pharmaceuticals, and the like.

1. An oral composition comprising:
reduced coenzyme Q10; and
at least one sesamin compound,
the oral composition having a mixing ratio by weight of the sesamin compound to the reduced coenzyme Q10 (sesamin compound/reduced coenzyme Q10) of 0.01 to 10.

2. The oral composition according to claim 1, further comprising theanine.

3. The oral composition according to claim 1,
wherein the oral composition has a mixing ratio by weight of the reduced coenzyme Q10 (X), the sesamin compound (Y), and theanine (Z) within a range surrounded by the following points in a three phase diagram:
(X, Y, Z)=(25, 75, 0), (25, 1, 74), (99, 1, 0).

4. The oral composition according to claim 1,
wherein the oral composition has a mixing ratio by weight of the reduced coenzyme Q10 (X), the sesamin compound (Y), and theanine (Z) within a range surrounded by the following points in a three phase diagram:
(X, Y, Z)=(25, 75, 0), (25, 1, 74), (75, 1, 24), (67, 33, 0).

5. The oral composition according to claim 1,
wherein the sesamin compound comprises at least one of sesamin and episesamin.

6. A method for producing an oral composition that contains reduced coenzyme Q10 and that exhibits reduced discoloration, the method comprising
mixing reduced coenzyme Q10 with at least one sesamin compound to give a mixing ratio by weight of the sesamin compound to the reduced coenzyme Q10 (sesamin compound/reduced coenzyme Q10) of 0.01 to 10.

7. The method for producing an oral composition according to claim 6, further comprising mixing the reduced coenzyme Q10 with theanine.

8. The method for producing an oral composition according to claim 6,
wherein the reduced coenzyme Q10 is mixed with the at least one sesamin compound or with theanine and the at least one sesamin compound to give a mixing ratio by weight of the reduced coenzyme Q10 (X), the sesamin compound (Y), and the theanine (Z) in the oral composition within a range surrounded by the following points in a three phase diagram:
(X, Y, Z)=(25, 75, 0), (25, 1, 74), (99, 1, 0).

9. A method for reducing discoloration of a composition containing reduced coenzyme Q10, the method comprising
mixing reduced coenzyme Q10 with at least one sesamin compound.

10. The method for reducing discoloration of a composition containing reduced coenzyme Q10 according to claim 9, further comprising mixing the reduced coenzyme Q10 with theanine.

11. A discoloration reducing agent for a composition containing reduced coenzyme Q10, comprising
at least one sesamin compound as an active ingredient.

12. The discoloration reducing agent according to claim 11, further comprising theanine as an active ingredient.

13. Use of at least one sesamin compound for reducing discoloration of a composition containing reduced coenzyme Q10.

14. The use according to claim 13, further comprising use of theanine.
